(19) United States
(12) Patent Application Publication
(10) Pub. No.: US 2024/0407674 A1
(43) Pub. Date: Dec. 12, 2024

(54) A DEVICE FOR MEASURING PHYSIOLOGICAL PROPERTIES

(71) Applicant: OMNIBUS 157 PTY LIMITED, Sydney (AU)

(72) Inventors: Kenneth Graham, Sydney (AU); Brody Payne, Sydney (AU); David Menzies, Sydney (AU); Karolina Petkovic, Sydney (AU); Mustafa Musaheh, Sydney (AU); Ricky Tjeung, Sydney (AU)

(73) Assignee: OMNIBUS 157 PTY LIMITED, Sydney (AU)

(21) Appl. No.: 18/696,839

(22) PCT Filed: Sep. 28, 2022
(86) PCT No.: PCT/AU2022/051158

§ 371 Date: Mar. 28, 2024

(30) Foreign Application Priority Data
Sep. 28, 2021 (AU) 2021903103

(51) Int. Cl. A61B 5/145; A61B 5/00; A61B 5/01; A61B 5/1477; A61B 10/00

(52) U.S. Cl. CPC A61B 5/14517; A61B 5/01; A61B 5/14546; A61B 5/1477; A61B 5/4266; A61B 10/0064; A61B 2560/0443; A61B 2562/028; A61B 2562/046; A61B 2562/125; A61B 2562/227

(57) ABSTRACT

A measuring device configured to provide an indication of at least one sweat property is provided. The measuring device includes a sensing device and a wearable device. The sensing device may comprise an inlet and at least one sensor. The inlet may be configured to allow sweat to enter the sensing device. The at least one sensor may be disposed at least one of at and adjacent to the inlet. The at least one sensor may be configured to sense a respective at least one sweat property. The wearable device may be connected to the sensing device. The wearable device may be configured to sense skin temperature. The sensing device may be disposable and interchangeable with another sensing device.

### TECHNICAL FIELD

The present invention relates to a device, and in particular, the present invention relates to a device for measuring one or more physiological properties such as one or more properties of sweat.

### BACKGROUND OF THE INVENTION

The following references to and descriptions of prior proposals or products are not intended to be and are not to be construed as, statements or admissions of common general knowledge in the art. In particular, the following prior art discussion does not relate to what is commonly or well known by the person skilled in the art, but assists in the understanding of the inventive step of the present invention of which the identification of pertinent prior art proposals is but one part.

In training for highly competitive sports or any physical training in general, knowledge of physiological properties such as heart rate and temperature, for example, can often assist in fine-tuning training programmes and improving competition performance. However, it is often difficult to obtain accurate measurements or indications of internal physiological properties which can further assist in customizing or improving training methods. As an example, if it could be determined whether an individual athlete is dehydrated based on their internal physiological signals, then certain actions can be taken in real-time to assist the athlete to improve their performance.

The present invention seeks to provide a device for measuring physiological properties such as one or more sweat properties, which may ameliorate the foregoing shortcomings and disadvantages or which will at least provide a useful alternative.

### SUMMARY OF THE INVENTION

According to one aspect, there is provided herein a sensing device configured to provide an indication of one or more sweat properties, the device including: an inlet, the inlet being configured to allow sweat to enter the device; and, one or more sensors disposed at or near the inlet, the sensors being configured to sense respective one or more sweat properties.

According to one example, the sensing device has two or more sensors disposed at or near the inlet, the sensors being configured to sense two sweat electrolytes respectively.

In another example, the sensing device has at least five sensors disposed at or near the inlet, the sensors being configured to sense five respective sweat electrolytes.

In a further example, the sweat electrolytes include Sodium ion (Na+), potassium ion (K+), Chloride ion (Cl−), Calcium ions (Ca++), and magnesium ions (Mg++).

In yet another example, the sensing device includes a secondary sensing mechanism for sensing sweat volume and/or sweat rate.

According to a further example, the secondary sensing mechanism is disposed near or next to the inlet.

In a further example, the secondary sensing mechanism includes a microfluidic channel.

According to another form, the microfluidic channel is disposed in a serpentine formation from the inlet to an outlet port.

In yet another example, the secondary sensing mechanism further includes an electrical impedance sensor.

According to another example, the electrical impedance sensor has a body aligned with the microfluidic channel, and two conductors connected to the body at an inlet end and to respective electric pads on the sensing device.

In another form, the two conductors are formed such that they are spaced-apart or electrically isolated from conductors joining the one or more sensors of the device to respective electric pads.

In yet another example, the wires are spaced-apart such that they are substantially parallel at the electric pads.

In a further form, the electric pads are disposed at or near an edge of the sensing device.

Thus, the sensing device can include a primary sensing mechanism which includes one or more sensors for sensing sweat electrolytes, and a secondary sensing mechanism for sensing sweat volume and/or sweat rate. The secondary sensing mechanism can include a microfluidic channel with electrodes/traces/conductors running parallel to the microfluidic channel to measure impedance and thus calculate sweat rate accordingly. Further, the device can include a series of electrical pads disposed along a length/edge/perimeter of the device for connecting to the primary and secondary sensing mechanisms. In one specific example, cross-over of the conductors from the primary and secondary mechanisms is limited.

In yet another example, the sensing device is formed from one or more layers or masks, each of the one or more layers or masks having separate sensing componentry disposed thereon.

According to a further example, the one or more layers or masks include any one or a combination of: a first layer having the inlet; a second layer having one or more openings for in fluid communication with the inlet and including one or more sensors for sensing respective one or more sweat electrolytes; a third layer having one or more secondary electrodes for sensing sweat volume and/or sweat rate; a fourth layer having a microfluidic channel.

In yet another example, the outlet port is a waste chamber and/or hydrophobic vent.

In a further example, the sensing device is configured to connect with a wearable device.

According to yet another example, the sensing device is a disposable device and interchangeable for another sensing device.

According to another aspect, there is provided a wearable device configured to communicate with a sensing device, the sensing device providing information in respect of sweat properties.

According to yet another aspect, there is provided a measuring device which includes a sensing device and a wearable device.

In yet another example, the sensing device is a disposable device and interchangeable for another sensing device.

According to a further aspect, there is provided herein a measuring device configured to provide an indication of one or more sweat properties, the measuring device including: a sensing device, the sensing device comprising: an inlet, the inlet being configured to allow sweat to enter the device; and, one or more sensors disposed at or near the inlet, the sensors being configured to sense respective one or more sweat properties; and, a wearable device connected to the sensing device, the wearable device being configured to sense skin temperature, wherein the sensing device is disposable and interchangeable with another sensing device.

It will be appreciated that the sensing device either separately or together with the wearable device can provide real-time sweat data through real-time continuous sweat sensing.

It will be appreciated that any of the features described in any of the sections herein can be combined.

### BRIEF DESCRIPTION OF THE DRAWINGS

The invention may be better understood from the following non-limiting description of a preferred embodiment, in which:

FIG. 1A is a top view of an example sensing device;

FIG. 1B is a top view of another example sensing device;

FIG. 1C is a photograph showing a top perspective view of an example of a sensing device;

FIG. 2A is a schematic of example electro-chemical componentry of an example sensing device;

FIG. 2B is a schematic of an example of electric pads of the example sensing device of FIG. 2A;

FIG. 3A is a schematic of another example sensing device;

FIG. 3B is a schematic of example ion sensors;

FIG. 3C is a schematic showing the ion sensors of FIG. 3B showing the tops of the ion sensing electrodes;

FIG. 3D and FIG. 3E are a schematic of an example of the tips of secondary electrodes;

FIG. 3F and FIG. 3G are a schematic of the secondary electrodes;

FIG. 3H is a schematic of an example of two secondary electrodes in parallel to each other;

FIG. 3I is a schematic of example electric pads connected to respective electrodes of FIG. 3H;

FIG. 3J and FIG. 3K are schematics of example microfluidic channels;

FIG. 4A is a schematic of a cross-sectional view an example sweat sensing device in accordance with an aspect of the present invention;

FIG. 4B is top perspective view of the example sweat sensing device of FIG. 4A;

FIG. 4C is a view from above the sensing device of FIG. 4A;

FIG. 4D is a view from below of the sensing device of FIG. 4A;

FIG. 4E is a cross-sectional view of the sensing device of FIG. 4A;

FIG. 5A is a schematic of a cross-sectional view an example sweat sensing device in accordance with another aspect of the present invention;

FIG. 5B is a cross-sectional view of the example device of FIG. 5A being unlocked/removed from a wearable device;

FIG. 5C is a view from above of the sensing device of FIG. 5A;

FIG. 5D is a view from below of the sensing device of FIG. 5A;

FIG. 5E is a cross-sectional view of the sensing device of FIG. 5A;

FIG. 6A is a schematic of a cross-sectional view an example sweat sensing device in accordance with another aspect of the present invention;

FIG. 6B is a view from above of the sensing device of FIG. 6A;

FIG. 6C is a view from below of the sensing device of FIG. 6A;

FIG. 6D is a cross-sectional view of the sensing device of FIG. 6A;

FIG. 7 is a schematic of an example of a sweat device communicating with other devices;

FIG. 8A is schematic of an example of a method of disposing ion sensors on a mask;

FIG. 8B is a schematic of an example of disposing impedance electrodes;

FIG. 8C is a schematic of an example removal of a photoresist;

FIG. 8D is a schematic of an example of deposition of an insulation layer;

FIG. 8E is a schematic of an example of photolithography and etching to open the insulation layer of FIG. 8D as required;

FIG. 8F is a schematic of an example of disposition of Ag electrodes;

FIG. 8G is a schematic of an example of the photoresist being removed;

FIG. 8H is a schematic of an example of a completed sensing device with a layer of silicone disposed on top;

FIG. 8I shows an example of how sweat can move through a sensing device that has been formed by the steps in FIGS. 8A to 8H; and,

FIG. 9 is a photograph of an example of two sensing devices formed on a substrate.

### DETAILED DESCRIPTION OF THE DRAWINGS

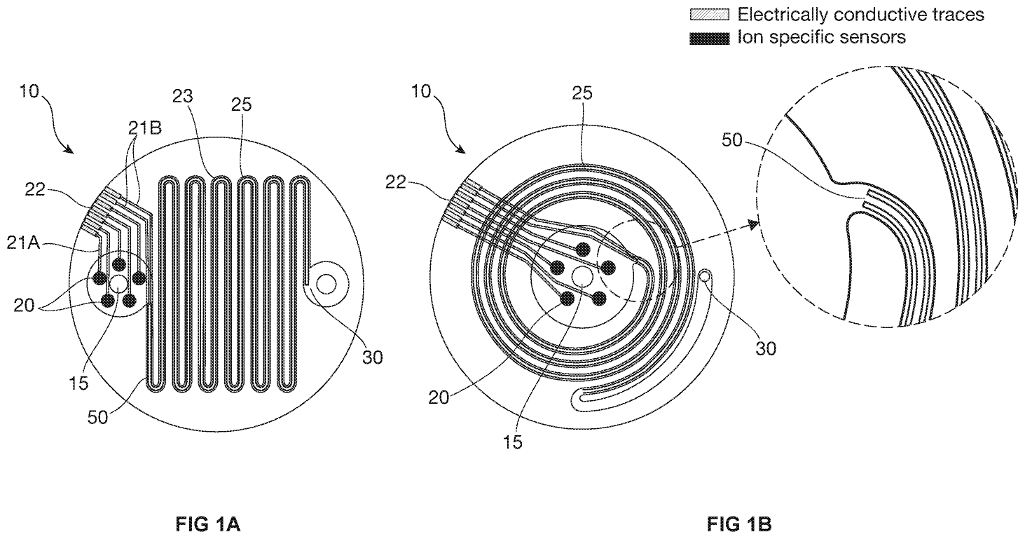

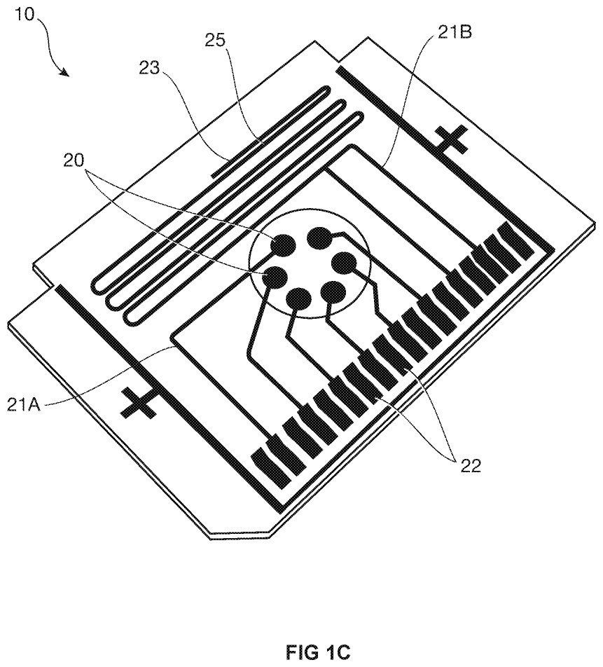

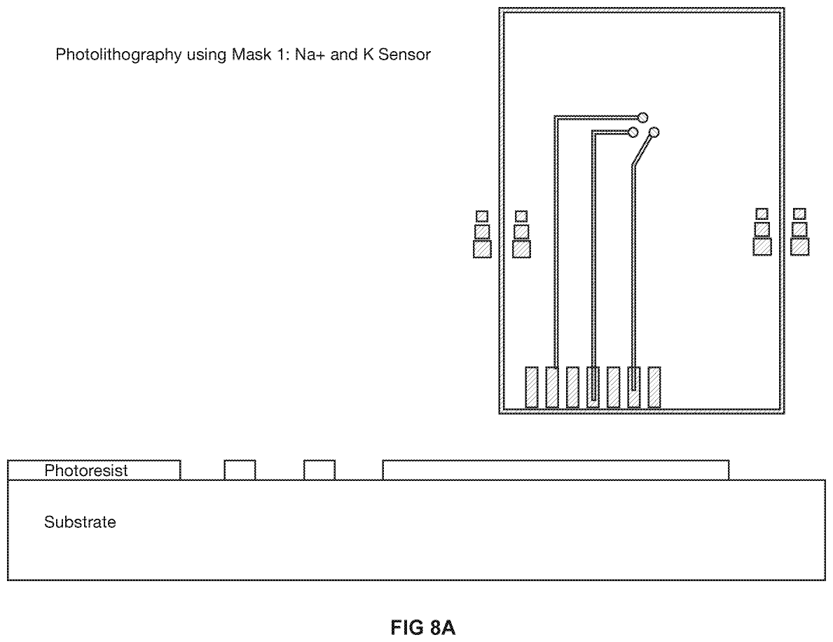

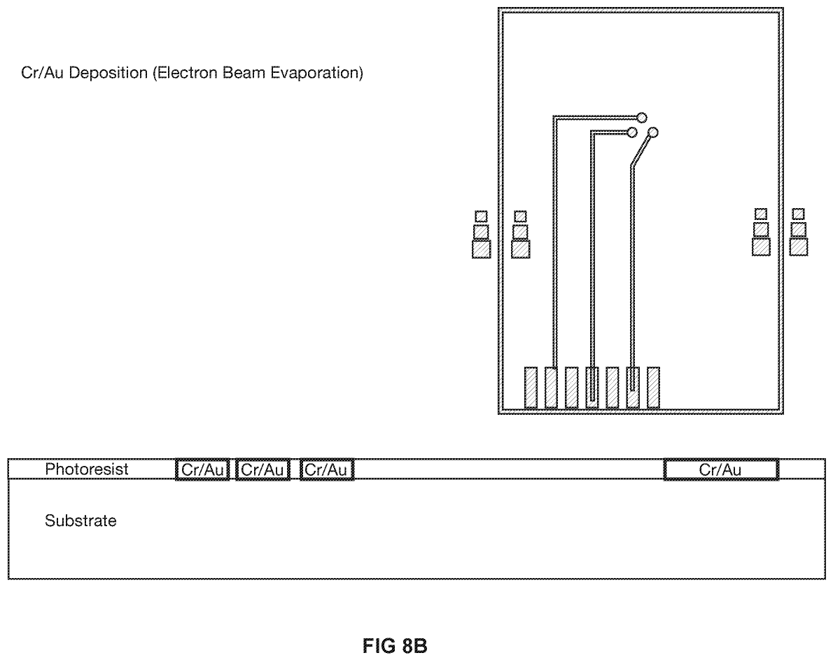

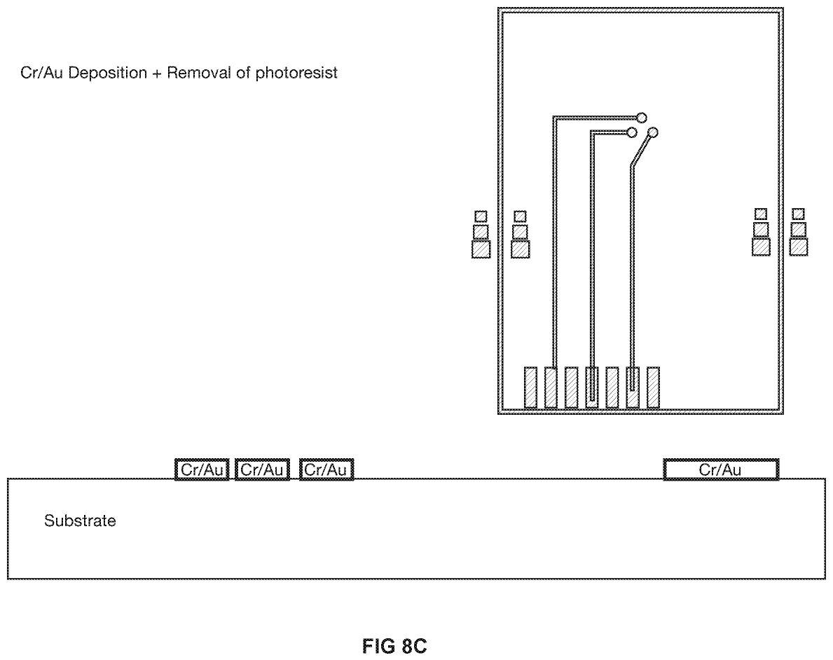

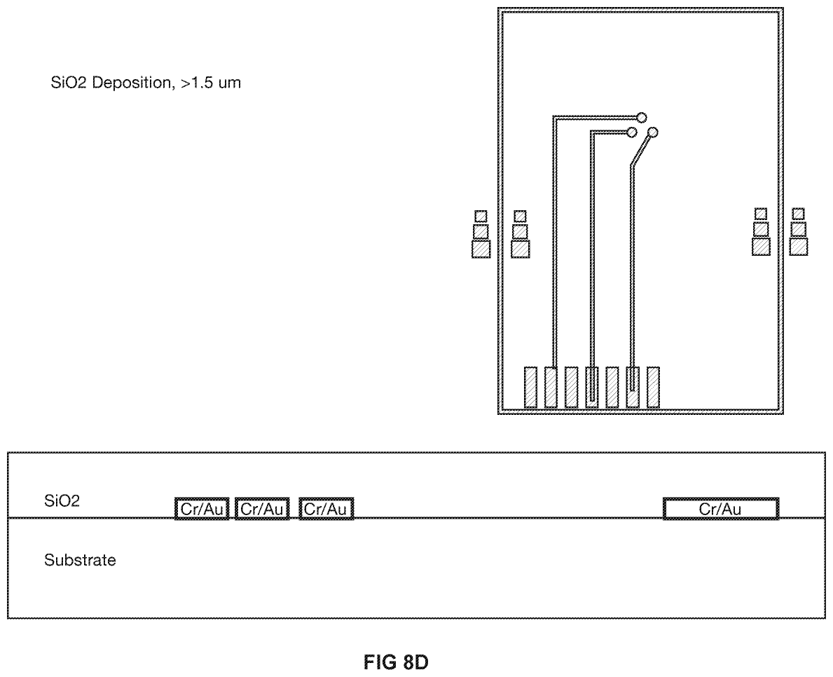

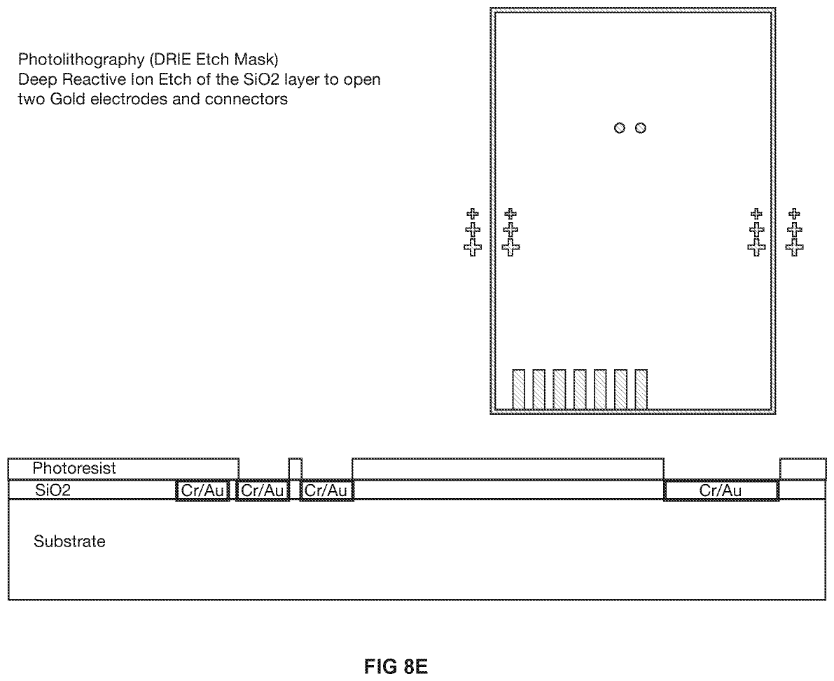

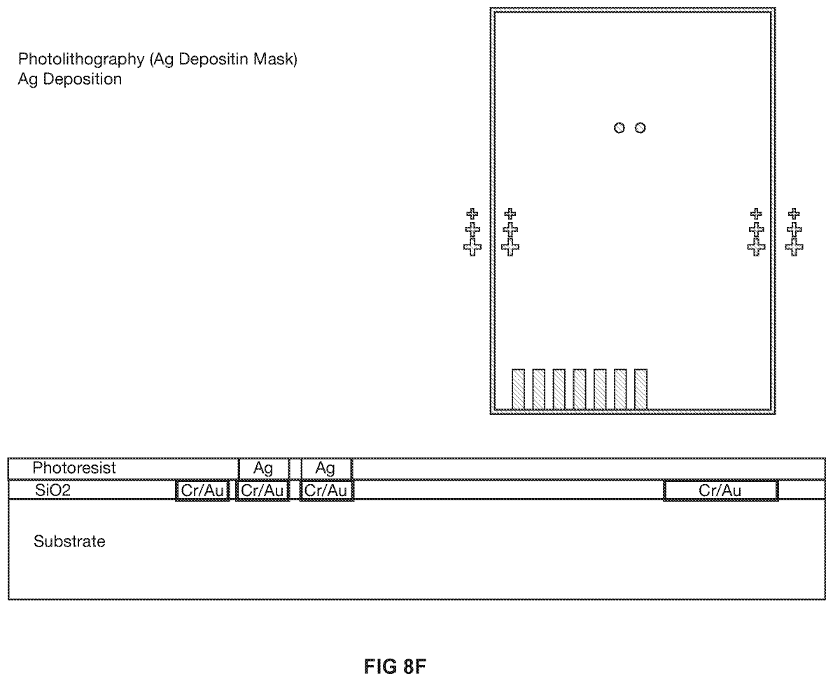

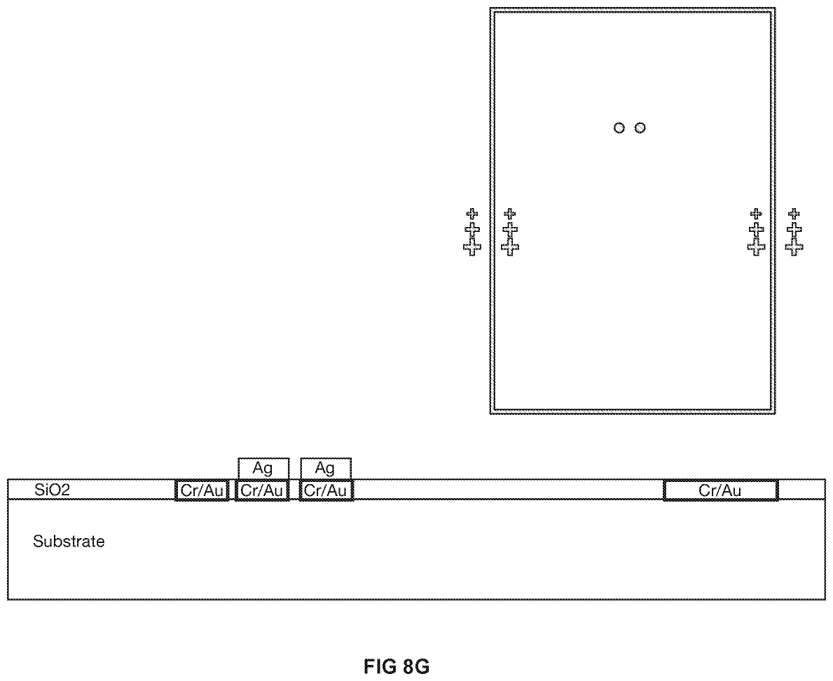

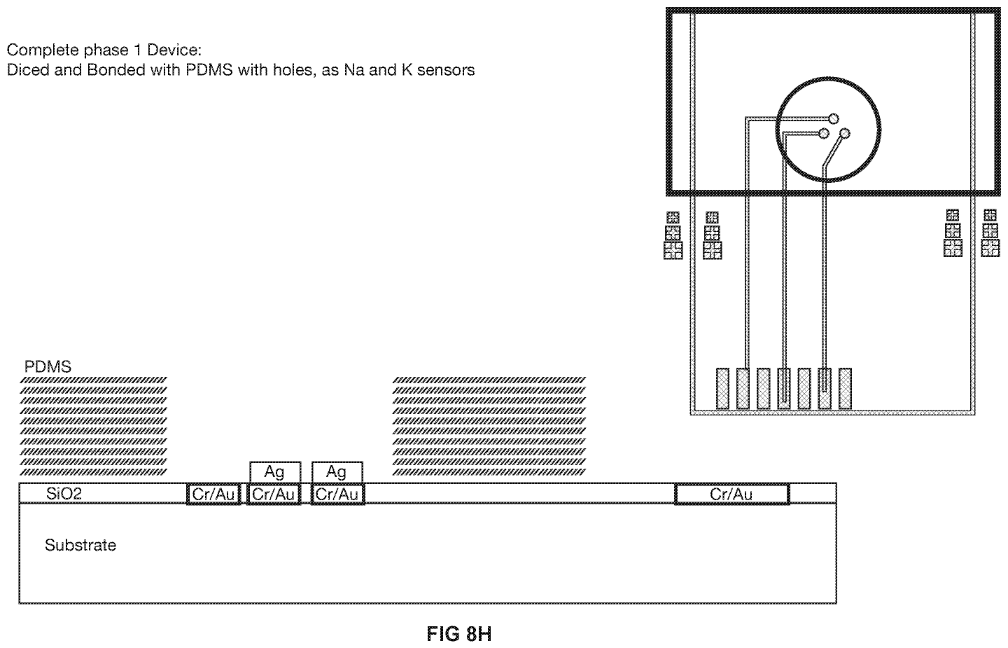

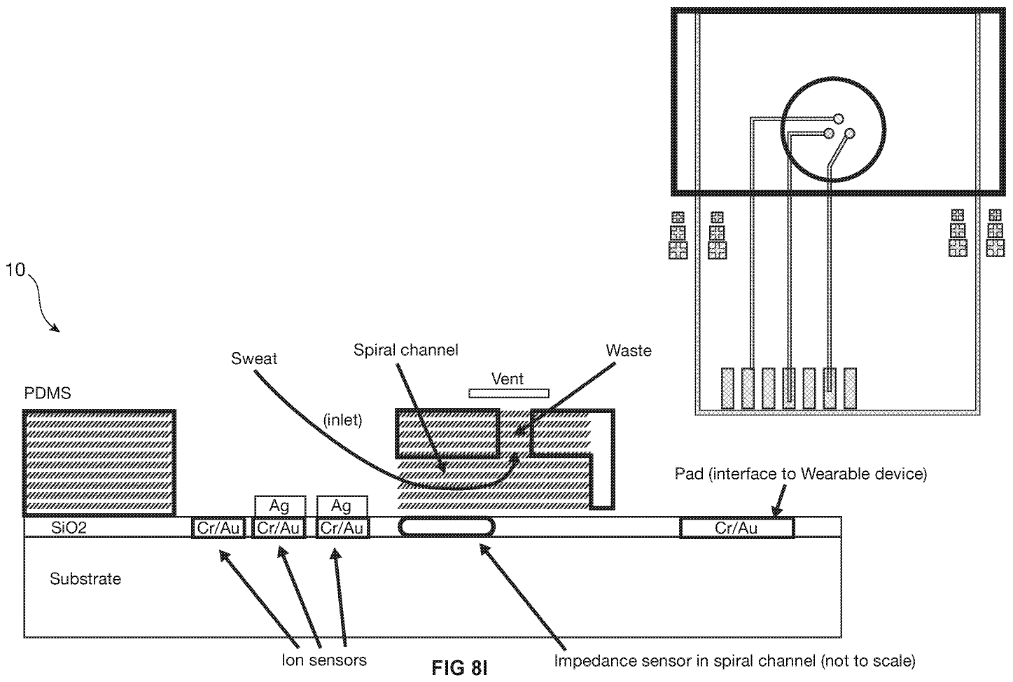

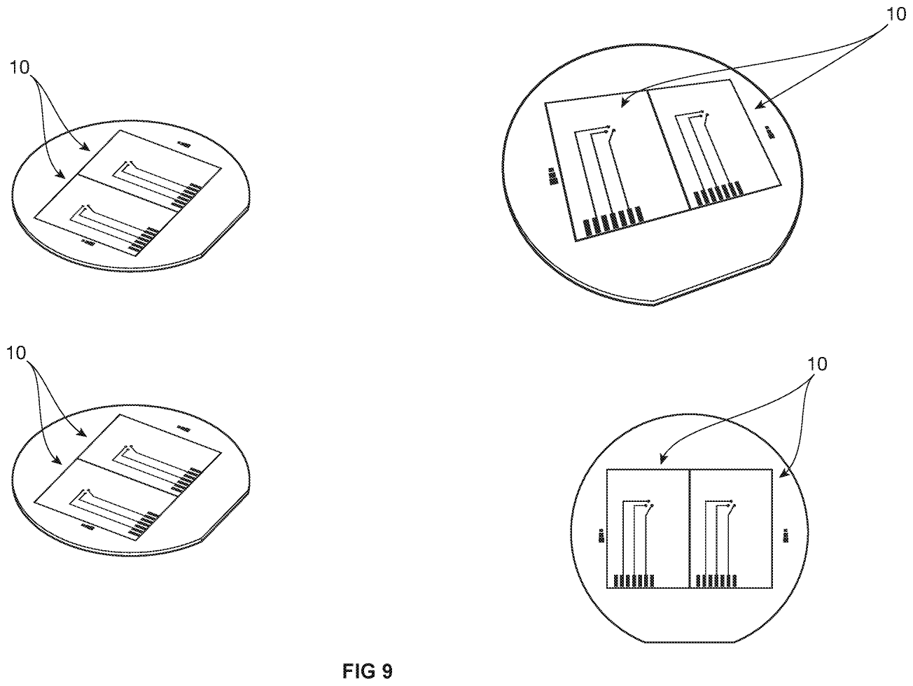

An example of a sensing device 10 described herein is shown in FIGS. 1A to 7 and further in FIGS. 8A to 9.

According to a particular example, as shown in FIG. 1A, the sensing device 10 includes an inlet 15 for receiving sweat, or where sweat can enter into the device 10. At or near the inlet 15 are one or more sensors 20, which are configured to sense one or more associated or corresponding sweat properties. It will be appreciated that the sweat properties can be any one or a combination of physio-chemical property such as electrolytes, hormones, metabolites, and the like.

In the example shown in FIGS. 1A and 1B, the inlet 15 is a circular chamber where there are five to six sensors 20 disposed around, and yet spaced-apart from each other within the inlet 15. In this example, the inlet 15 is circular and thus the sensors 20 are disposed concentrically around the inlet 15. In this particular example, each of the sensors 20 are electro-chemical sensors for sensing specific ion, for example, Sodium ion (Na+), potassium ion (K+), Chloride ion (Cl−), Calcium ions (Ca++), and magnesium ions (Mg++), and can also include a reference sensor/electrode. It will be appreciated that anywhere from one to five or more than five ions can be sensed as required with additional electrodes/sensors.

The one or more sensors 20 are typically connected to corresponding one or more electrical traces or pads 22, which provide the electrical connection between the sensing device 10 and a wearable device 100, which is further described below. The plurality of sensors 20, together with their respective electrodes/wires form a primary or ion sensing mechanism.

FIGS. 1A and 1B further show that the device 10 can include a secondary sensing mechanism, including a microfluidic channel 25 which is configured to carry/transport the sweat away from the inlet 15 to an outlet port 30. Thus, for example, as sweat fills up the inlet 15, it is typically then transported along the channel 25 and continuous sweat secretion will typically mean that the sweat in inlet 15 is continuously replenished.

Notably, the microfluidic channel 25 can be disposed in any matter at, near, or around the inlet 15. FIGS. 1A and 1C show an example where the channel 25 is disposed in a serpentine manner/formation next to or proximate the inlet 15. FIG. 1B shows the channel 25 being disposed in a spiral around the inlet 15, spiralling from the inlet 15 outwardly to the outlet port 30.

It will be appreciated that the outlet port 30 is typically a waste chamber and/or a hydrophobic vent and can prevent pneumatic backpressure slowing or stopping fluid movement through the microfluidic channel 25.

Further to the microfluidic channel 25, a sweat rate sensor (that is, an electric impedance sensor) 35, typically runs in parallel or is aligned with the microfluidic channel 25. The sweat rate sensor typically includes two parallel electrical wires or conductors 50 (described further below) which are disposed on either side of the microfluid channel such that as the microfluidic channel collects the volume of sweat and, the sweat rate sensor 35 measures this volume by changes in impedance (increased fluid volume reduces impedance) across the channels of the sensor 35. That is, the measured current is a function of volumetric flow rate or sweat rate in this particular example. The sensor 35 is typically aligned with or runs along the length of the channel 25 and in contact with the fluid/sweat inside the channel 25. However, it will be appreciated that the geometry or shape of the channel 25 and sensor 35 may vary (as shown for example in FIGS. 1A, 1B, and 1C).

It will be appreciated that the sensing device 10 can thus allow the monitoring of sweat constituents (such as, in this example, at least the five sweat ions Na+, K+, Cl−, Ca++, and Mg++) through the electrochemical sensors 20, and sweat rate through the secondary sensing mechanism including the microfluidic channel 25 and the electrical impedance sensor 35.

Referring more specifically to FIGS. 1A and 1C, where the microfluidic channel 25 is formed in a serpentine manner or formation, the microfluidic channel is disposed in a serpentine formation from the inlet 15 to the outlet port 30, the electrical impedance sensor 35 typically includes two electrodes/conductors (referred to as secondary electrodes 50 herein) with a body 23 each that are aligned substantially with the microfluidic channel 25, and disposed at either side of the channel. The impedance sensor 35 also includes two wire legs 21B which are connected to the respective body 23 at an inlet end where the microfluidic channel 24 terminates and the two wire legs 21B connect to respective electric pads 22 on the sensing device 10.

Thus, it will be appreciated that in this example, the two wire legs 21B are formed such that they are spaced-apart or electrically isolated from wires (or electrical connections/conductors) 21A joining the one or more sensors 20 of the device 10 to respective electric pads 22. Accordingly, the wires (or electrical connections) 21A and 21B are spaced-apart such that they are substantially parallel at the electric pads.

It will be appreciated that in the examples shown in FIGS. 1A and 1C, the secondary sensing mechanism is formed in a serpentine manner. However, other formations may also be possible such that the two wire legs 21B from the secondary sensing mechanism do not cross over with wires 21A from the sensors 20 of the primary sensing mechanism or such that crossing over between the wires is limited.

Typically, the electric pads 22 are disposed at or near an edge of the sensing device 10, and can be disposed in a series along the edge or perimeter of the device 10. In these examples, the sensing device 10 is formed such that there is a series of electrical pads 22 which connect to respective electrodes of different componentry. The way in which the primary and secondary sensing mechanisms are formed on the device 10 is such that the electrodes which connect to the electrical pads 22 do not cross-over or cross-over is limited thereby minimising any interference or noise between the plurality of electrodes.

It will be thus appreciated that such a formation whereby crossing over of the conductors/wires from the primary and secondary mechanisms are limited, can allow for the primary and secondary sensing mechanisms to be disposed on the same substrate layer of the device 10 or minimise the layering between the componentry. Furthermore, the formation can allow for a plurality or more sensing electrodes to be disposed on the substrate (for example, as shown in FIG. 1C, 5 sensors and a reference electrode).

The ion sensors 20 and sweat rate sensor 35 and the microfluidic channel 25 are further described below.

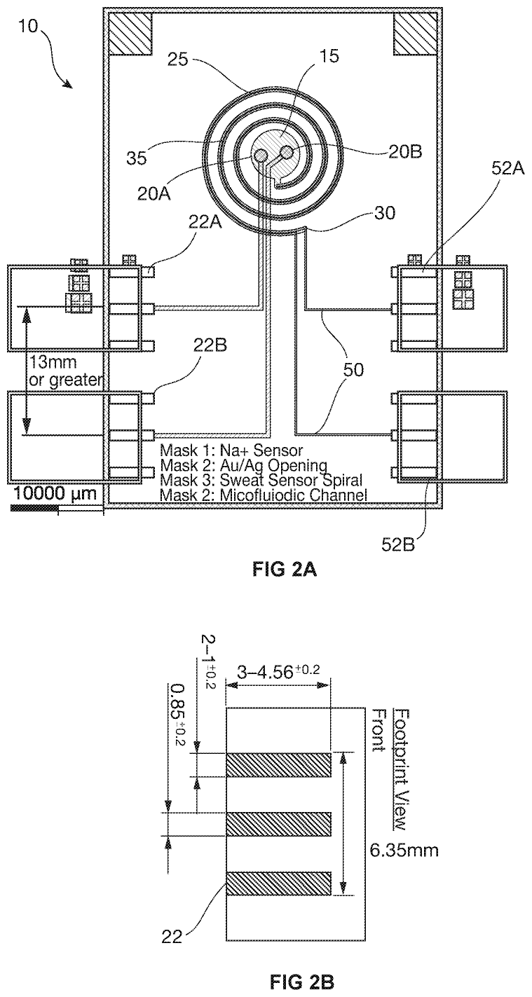

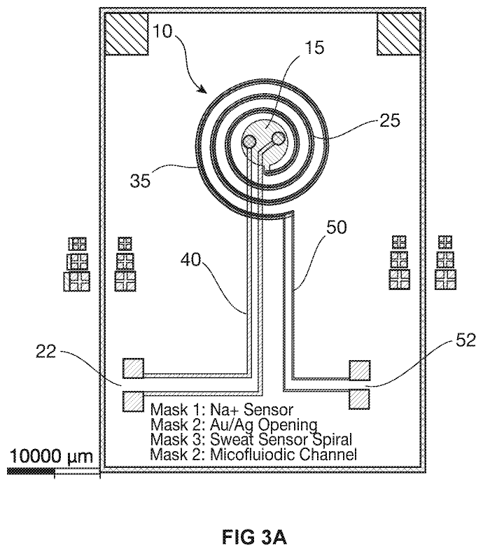

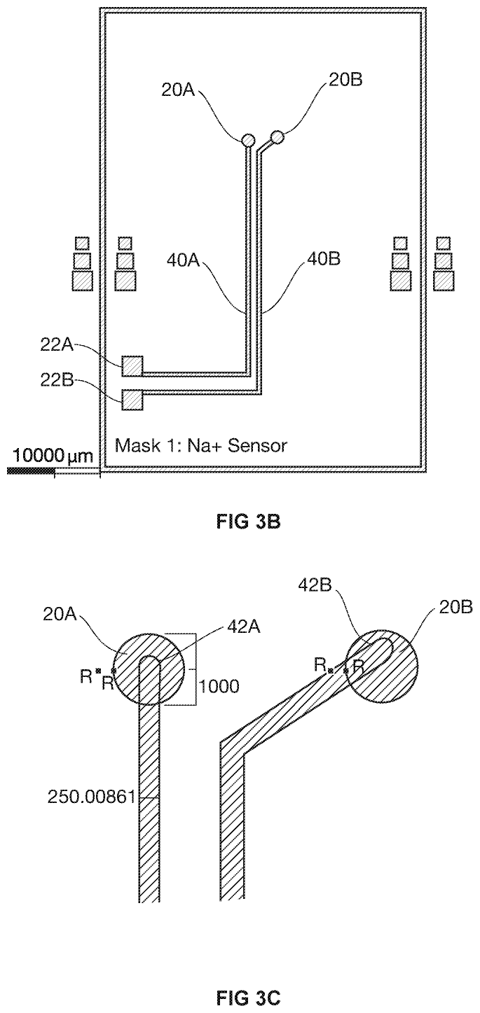

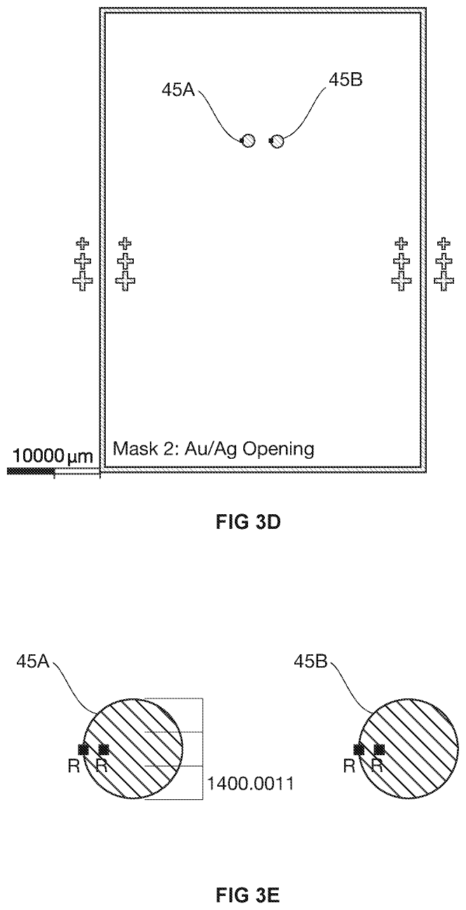

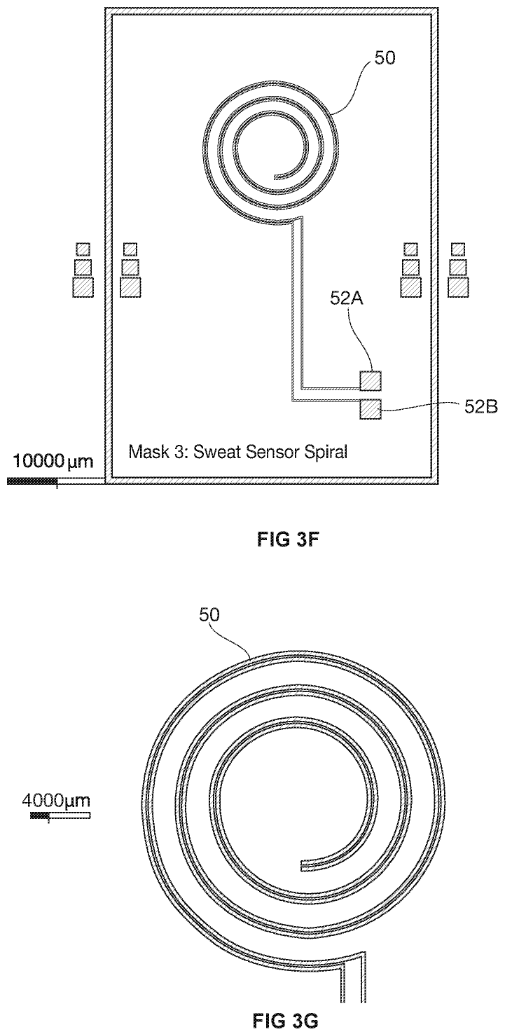

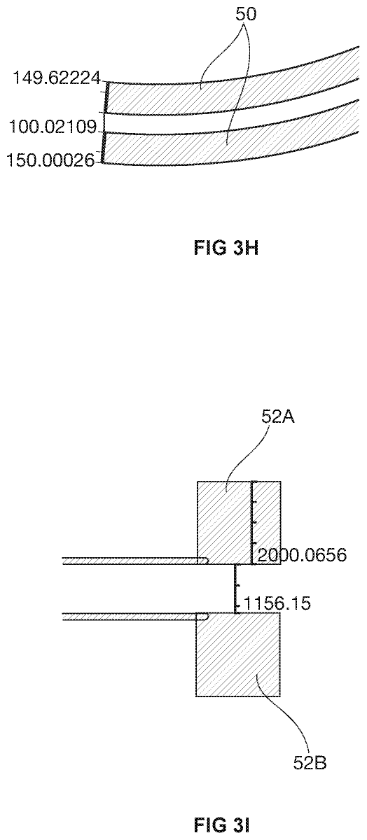

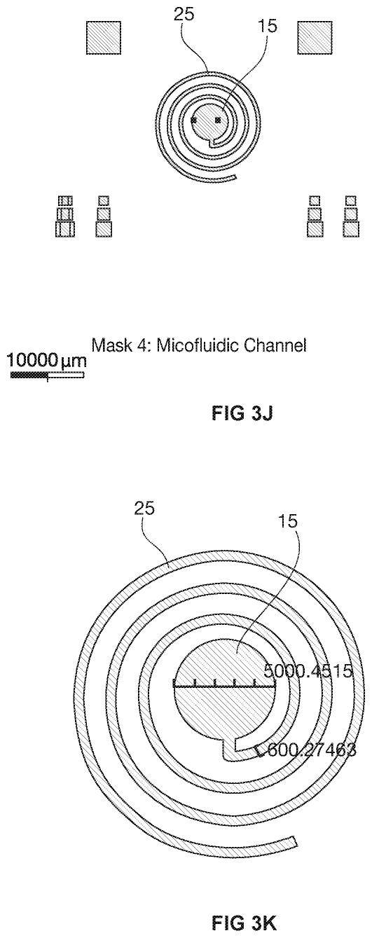

A further example of the sensing device 10 is shown in FIGS. 2 to 3K. In these examples, FIGS. 2A and 3A show different examples of the components of the sensing device 10. In particular, the example of the sensing device 10 in FIGS. 2A and 3A show an example of the sensing device 10 being configured to sense two ions, where the two ion sensors 20A, 20B are electrically connected to corresponding pads 22A, 22B.

FIG. 2B shows an example of the electrical pads 22A/22B which can be used. In this particular example, the electrical pads 22A/22B are connectors which can, for example, be mounted/slotted onto the edge of a PCB (Printed Circuit Board) or the like.

FIGS. 3A to 3K are examples of the different layers or masks and components of the sensing device 10 which are typically disposed or overlayed as layers/masks to form the sensing device 10 as shown in FIG. 2A.

Referring more specifically to FIG. 3B, FIG. 3B shows a first layer of the sensing device 10 where there is located thereon electrical connections 40A, 40B connecting the ion sensors 20A, 20B respectively to corresponding pads 22A, 22B. FIG. 3C shows the tips 42A, 42B of the electrical connections 40A, 40B. The ion sensors 20A, 20B are typically electrochemical sensors which can generate an electrical signal based on the presence of a particular ion, which is then typically picked up at the pads 22A, 22B. Notably, the electrical connections 40A, 40B can be vias which can also be formed in a bridge or the like to pass the microfluidic channels 25 in order to minimise any cross-contamination between the electrochemical sensors and the sweat rate sensor.

FIGS. 3D and 3E show an example of a second mask with one or more openings 45A, 45B. Typically, the openings 45A, 45B are in fluid communication with the inlet 15 and the tips 42A, 42B of the respective electrical connectors 40A, 40B are disposed within respective openings 45A, 45B. Thus, sweat entering the inlet 15 can be sensed by particular ion sensors 20A, 20B which have respective openings 42A, 42B in fluid communication with the inlet 15 and the electrical signal can be detected by the electrical connectors 40A, 40B accordingly.

FIGS. 3F and 3G show a third layer comprising of one or more traces or secondary electrodes 50. These are connected to secondary pads 52A, 52B respectively (which have been described above as pads 22). FIG. 3H shows an example section of the traces 50 and FIG. 3I shows an example of the secondary pads 52A and 52B.

Notably, in the examples shown, FIG. 3B shows the two ion specific electrodes 20A, 20B as terminating at position 22A and 22B (typically electrical pads disposed to communicate with a wearable device 100). That is, 20A and 20B connect electrically to 22A and 22B via electrodes or traces 40A, 40B respectively. The two parallel impedance electrodes 50 terminate at 52A and 52B. The microfluidic channel 25 effectively or includes the impedance electrodes 50, which terminate at 52A and 52B, which provide electrical communication with the wearable device 100.

According to one particular example, the electrodes 50 are typically silver (Ag), gold (Au), and/or chromium (Cr) parallel electrodes and sweat rate can be measured by measuring the magnitude of impedance between the two electrodes 50. Further, the sweat collection tube or microfluidic channel is of a known diameter, and thus the volume of sweat collected will be πr²×length. Accordingly, the impedance measured between the two electrodes 50 will typically decrease with increasing fluid volume due to the decrease in resistance and increase in capacitance.

FIGS. 3J and 3K show further examples of the microfluidic channel 25 which is typically disposed on a fourth layer or mask. The masks/layers are then overlaid to form the sensing device 10.

It will be appreciated that although 4 masks/layers are described herein, any number of masks/layers can be used to give effect to the sensing devices. As an example, there may be an insulation layer or mask between the different sensing layers to improve functionality and decrease or limit any cross-contamination between different signals being sensed. Alternatively, both the electrochemical sensors 20 and the impedance sensor 35 can be provided on the same layer/mask.

Notably, the sensing device 10 can be a part of, be connected to, or be integral with a wearable device 100. Together, the sensing device 10 and the wearable device 100 can form a measuring device 90. In one particular example, the sensing device 10 is a disposable part of the wearable device 100. Thus, according to one particular example, the sensing device 10 can measure particular sweat properties as required, the signal for which can be transmitted to the wearable device 100 for processing, display, and the like.

Accordingly, as a user uses the wearable device 100 and if the device 10 is a disposable part, the user can interchange the sensing device 10 as required.

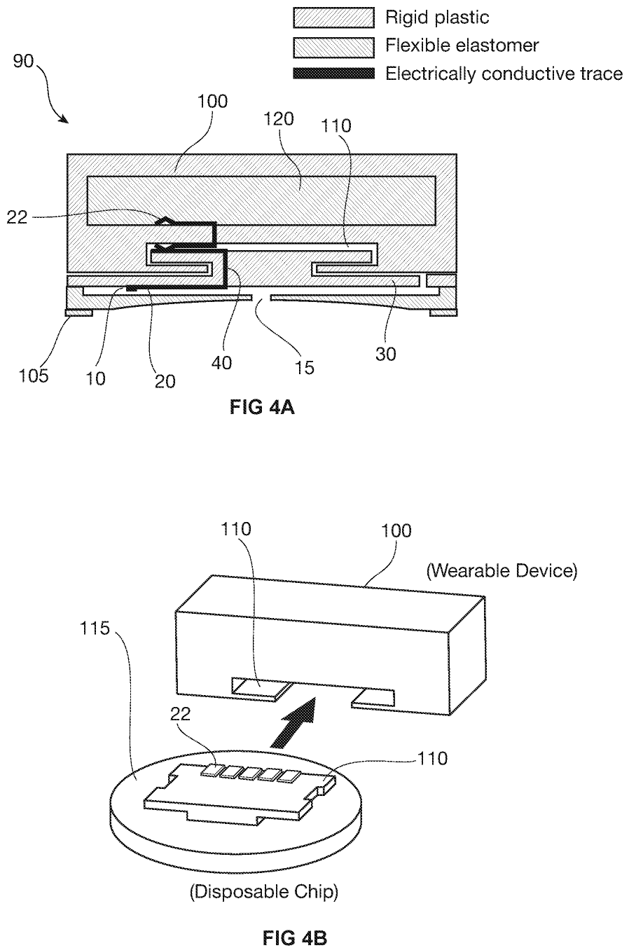

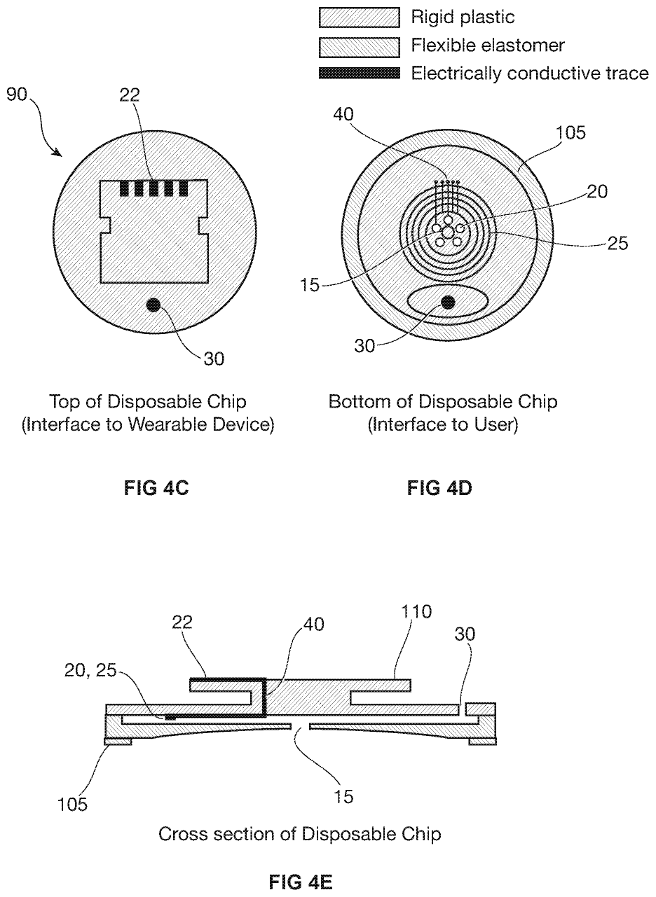

Examples of the wearable device 100 together with the sensing device 10 is shown in FIGS. 4A to 7.

According to one example, FIG. 4A to 4E show how the sensing device 10 can be connected to or received by a wearable device 100. In this particular example, the sensing device 10 is connected to the wearable device 100 at a portion where the pads 22 are located as the pads 22 are configured to transfer relevant electrical signals for analysis to the device 100. In the example of FIGS. 4A to 4E, the pads 22 are formed/disposed on an elevated portion 110 of the device 10. The elevated portion 110 in this example has a T-shaped cross-section which is configured to slide into a corresponding female portion in the wearable device 100 and clicked or locked/held therein. Thus, for example, the elevated portion 110 can be a T-slot mechanical interface for the device 10 to the wearable device 100. It will be appreciated, however, that the elevated portion 110 may be part of (that is co-planar with) the surface 115 of the device 10.

Referring more particularly to FIG. 4A, which shows an example cross-section of the sensing device 10 inserted into the wearable device 100, the FIG. 4A shows how different layers of the measuring device 90 are formed such that electrical signals from the sensing device 10 can be received by the wearable device 100 and analysed accordingly. In this example, a base layer 105 typically includes an adhesive or the like for adhering the device 10 to a user. Sweat then typically enters the device 10 through the inlet 15 and is sensed by the sensors 20 which through an electrical/chemical connection 40 is picked up by pads 22, which are electrically connected via the T-slot mechanical interface 110 to the wearable device 100. The device 10 can include the masks/layers described herein, including the outlet port 30, which is typically a waste chamber/hydrophobic tent. Notably, at 120, the wearable device 100 includes electronics/componentry such as batteries, PCB, antennas, and the like.

It will be appreciated that the example shown in FIGS. 4A to 4E can provide a robust connection between disposable device 10 and the wearable device 100 through a simple connection, such as a click detent, which typically has good usability and feedback (such as through a clicking sound or the like).

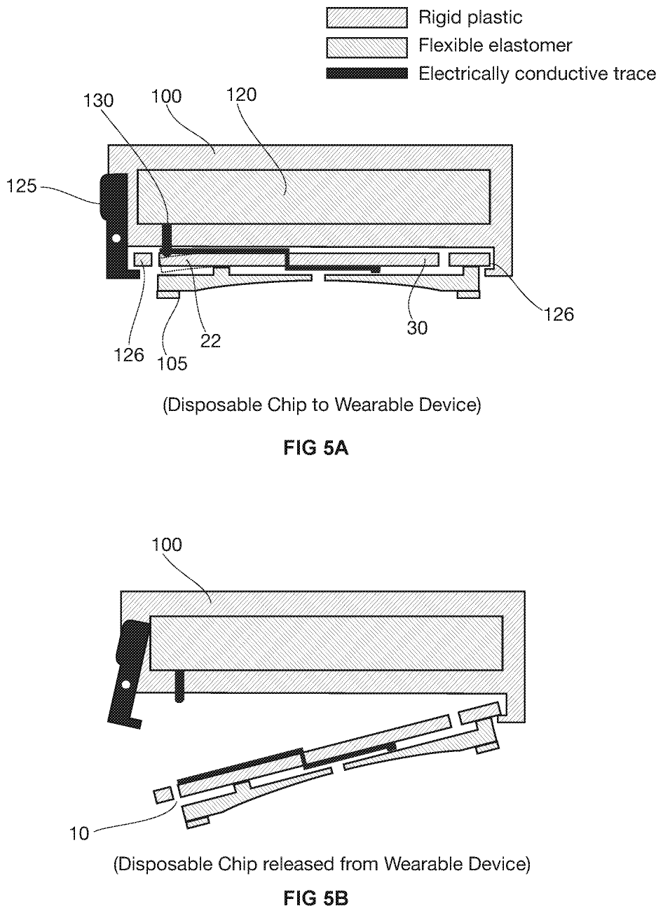

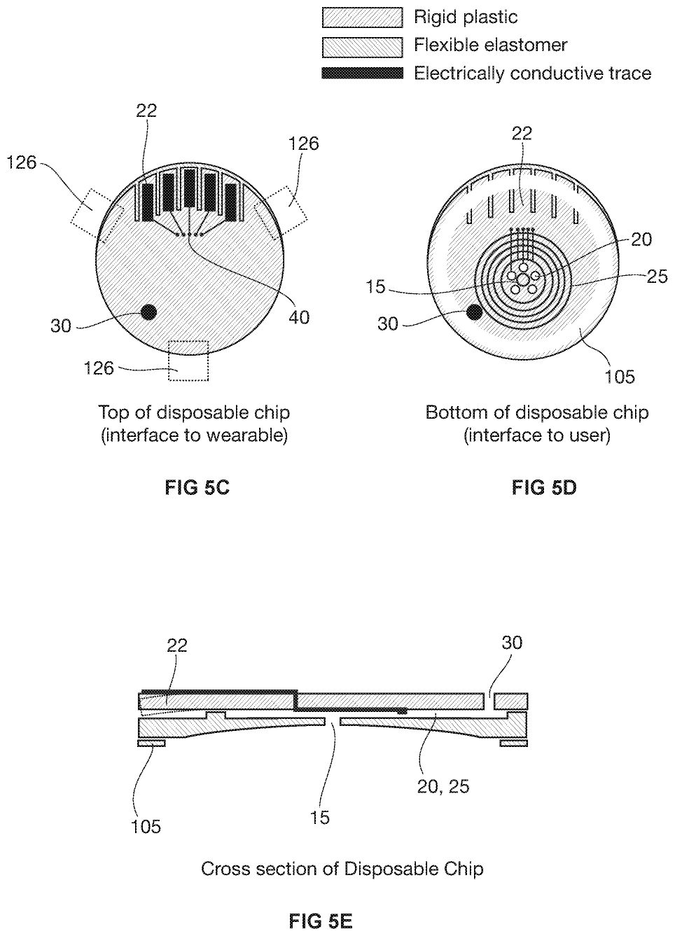

FIGS. 5A to 5E show a further example of a wearable device 100 together with a sensing device 10. In this particular example, the sensing device 10 snap-locks into the wearable device 100 and is held in place by a locking mechanism 125. In this particular example, the locking mechanism 125 is a spring-loaded mechanism which can snap-lock the sensing device 10 into place within the wearable device 100. The sensing device 10 has one or more edges or flanges 126 which can engage with the locking mechanism 125 such that the sensing device 10 can be held in place to allow for electrical connection between the two devices 100 and 10. The sensing device 10 can then easily be ejected/disconnected from the wearable device 100 and disposed of accordingly. In this particular example, the wearable device 100 also includes an electrical contact 130 which can be connected to the pads 22 to provide an electrical connection between the wearable device 100 and the sensing device 10. Thus, in this particular example, electrical contact compliance can be built into the sensing device 10 which can allow for a more simplified waterproofing between the devices 10 and 100. Further, the device 100 can be easy to clean and the sensing device 10 can be loaded and ejected (thus disposed) simply from the wearable device 100.

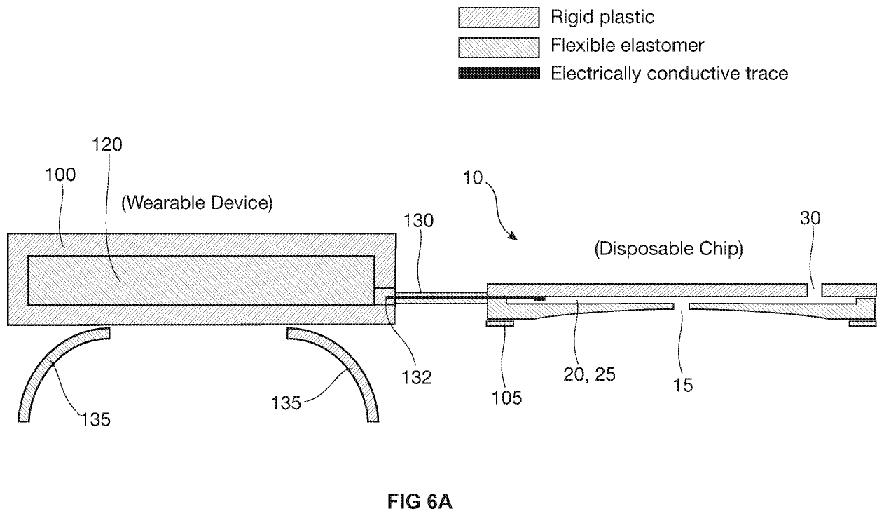

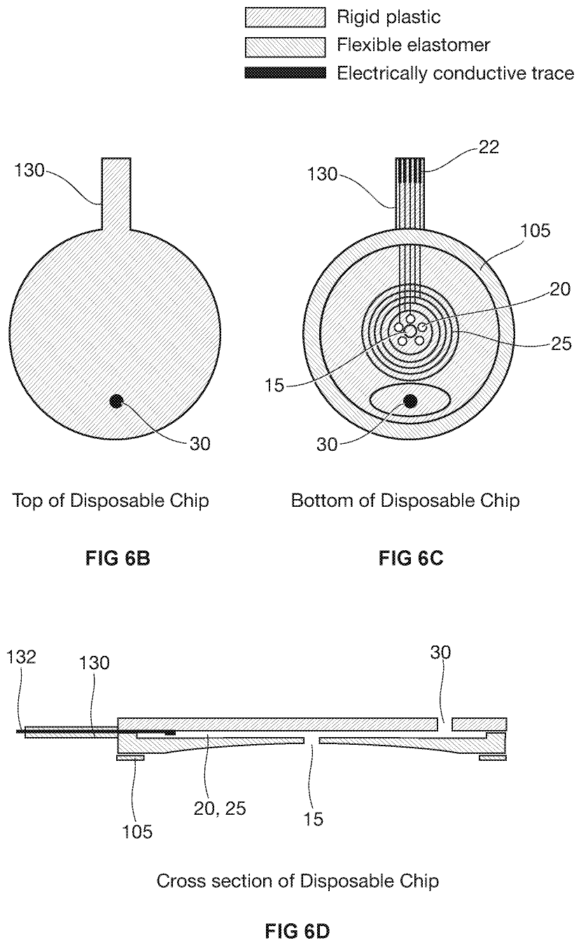

FIGS. 6A to 6C show another example of a wearable device 100 connected to a sensing device 10. In this particular example, the wearable device has one or more bands or straps 135 that can connect the wearable device 100 to a user, such as for example, a user's wrist, chest, leg or the like. Thus, the device 90 can be designed to be attached to the skin of a user with either an adhesive patch and/or strap that wraps around the device and the limb.

Further, the examples in FIGS. 6A to 6C show the sensing device 10 being connected to the wearable device 100 via an electrical contact 130 which is a lead as the sensing device 10 sits next to the wearable device 100. Thus, for example, the sensing device 10 can be a disposable patch or chip that can have an adhesive base layer 105 that can adhere to a portion of skin of the user, next or proximate to where the wearable device 100 is attached to the user. The lead 130 extends between the wearable device 100 and the sensing device 10 and connects typically via an edge connecter 132 or the like. In this example, the electrical pads 22 can be at the end of the lead (ribbon or cable) 130 which connects directly to the wearable device 100. Typically, the lead 130 and the edge connector 132 is waterproof.

The particular example shown in FIGS. 6A to 6C can simplify the sensing device 10 as this particular example does not require the use of vias. Further, there is a lower physical or vertical profile to device 90 overall given the sensing device 10 and the wearable device 100 are not stacked. Additionally, as the sensing device 10 is exposed, it can be more readily replaceable without the requirement of taking the wearable device 100 off. The connection 130 between the sensing device 10 and the wearable device 100 can be made of a flex printed circuit which can also allow strain relief between the connections.

It will be appreciated that there is provided herein a measuring device 90 that can be worn by a user and that is configured to collect and analyse sweat volume and composition from a known area of skin. The device 90 can wirelessly transmit data received/generated by the device 90 to a processing system and allow the calculation of sweat rate, rate of the loss of electrolytes for the measurement site to allow the calculation of whole-body fluid and electrolytes loss.

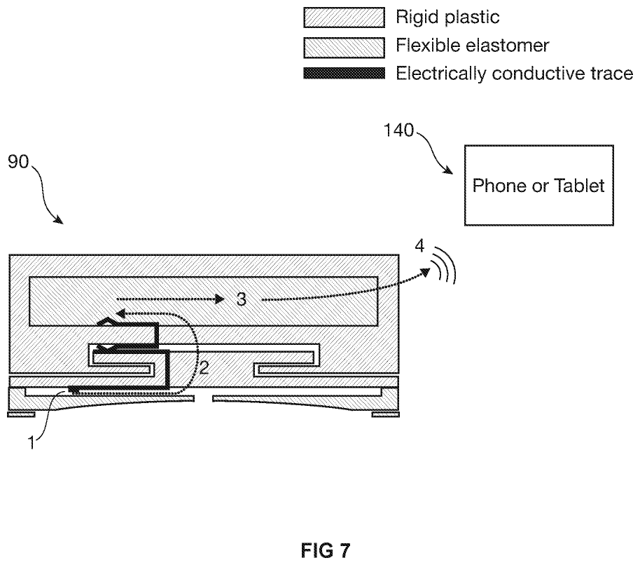

FIG. 7 shows an example of a sweat device 90 which typically comprises of the wearable device 100 and the sensing device 10, which is able to communicate with a remote device such as a computer or processing system 140, and in one example, this can be a tablet or telecommunication device/phone.

In the example shown in FIG. 7, an analogue voltage signal is generated by the ion specific sensors (at 1.) when they come into contact with the target anolyte (Na+, K+, and any other ion being sensed). The analogue signal is typically then recorded, filtered and possibly amplified by wearable device 100 (at 2.). The wearable device electronics system can then convert this analogue signal to digital (at 3.). Notably, the wearable device may perform some data processing or analysis on board and then the wearable device can transmit the digital data to a phone, tablet, or the like, via Bluetooth or any other wireless signalling means (at 4.). The phone/tablet software application can manipulate and analyse the data and can provide feedback to the user accordingly.

Accordingly, the device 90 can typically comprise of:

1. a disposable component 10 which has an adhesive surface to adhere to the skin and which also contains a collection orifice 15, ion specific electrodes (20, 22) and calibrated microfluidic channel 25, together with an impedance sensor 35, and electrical connectors/pads to a wearable component; and,
2. a non-disposable waterproof wearable component 100 containing processing system(s) to control data flow, data storage, calibration data, a skin temperature measurement module, a wireless communications module and a power source for operation.

Notably, the disposable component is typically a single use component as the act of measurement typically can cause degradation of the electronics due to sacrificial nature and, the challenges of cleaning out of the microfluidics channel to remove residual salts and fluid from sweat that has been previously measured. However, it will be appreciated that the sensing device 10 and the wearable device 100 can be formed such that they are integral to one another and the device 10 is non-disposable or not disposable in its entirety.

According to one example, the sweat collection elements are located on the disposable component 10 and sweat collection is via a circular orifice 15 that has a lip or the like on it so that the skin collection area can be sealed so only fluid from sweat glands within the area of the lip is collected. Notably, the device 10 can also include a pressure sensitive adhesive at its base either together or separate to the lip to allow for a designation/determination of a sweat collection area.

As described herein, the centre of the orifice/inlet 15 also has microfluidic channel 25 of a specific diameter, such as, for example, 600 micrometres wide and 200 micrometres deep that is open on the distal end, ending at the outlet port 30, leading away from the inlet 15 that collects sweat via a combination of capillary action and low-pressure hydraulics. Thus, for example, the sweat typically moves along the microfluidics channel by capillary attraction of the face of the fluid-air boundary and that sweat is being actively pushed out of the sweat glands and along the microfluidics channel (hydraulics).

The proximal opening of the microfluidic channel, at the inlet 15 has an array of ion specific electrodes 20 that can measure the concentrations of the electrolytes at the entry of the sweat fluid into the orifice 15.

The microfluidic channel 25 also has flowrate electrodes via the secondary electrodes 50 along its length to provide a signal when the fluid has reached them to allow fluid volume and hence flow rate to be calculated.

Notably, the disposable component 10 can also have connectors 22 allowing for power and data flow to and from the non-disposable component.

The wearable device 100 can also include:
a skin thermistor or infra-red thermometer to measure skin temperature under the disposable component;
a memory chip to allow for data storage for later downloading later or for real-time transmission;
a wireless communication module for receiving calibration data and system upgrades and, also for streaming data to a base station, tablet, or mobile phone;
a rechargeable system power source; and,
processing electronics.

It will be appreciated that the sensing device can be made by creating the different masks/layers as required. According to one specific example, the different masks/layers can be produced by nanofabrication through:

1. Fabrication of Na+ sensor with Au/Ag opening.
Design and fabricate Mask 1 (Na+ sensor) and 2 (Au/Ag opening).
Microfabrication of Na+ sensor with Au/Ag opening
No dicing, 2 devices per glass wafer, work to be done on 4 wafers.
Using SiO2 as insulation layer.
Bonded with Blank PDMS with a single (punched) hole opening.
   2. Fabrication of Na+ sensor with Au/Ag opening.
2 devices per glass wafer.
Using SiO2 as insulation layer
   3. Fabrication of the Sweat sensor spiral.
Design and fabricate Mask 3 (Sweat sensor spiral).
Microfabrication the Sweat sensor spiral, continuing the work above.
Dicing to individual device
   4. Fabrication of Microfluidics channels.
Design and fabricate Mask 4 Microfluidics channels.
Master Mould Fabrication using SU-8 on Silicon wafers
PDMS (Polydimethylsiloxane) Replica Fabrication
Bonding to individual device

Further examples of nanofabrication steps for placing various electrochemical componentry as described herein on each layer/mask is shown in FIGS. 8A to 8H. In these examples, the various steps of the disposition of the sensors and electrodes are shown, including disposing of insulation layers, and the like, as required. For example:

1. Photolithography is used to dispose ion sensors Na and K on a mask, as shown in FIG. 8A;
2. Electron beam evaporation is used to dispose the impedance electrodes (Cr/Au), as shown in FIG. 8B;
3. FIG. 8C shows removal of the photoresist;
4. FIG. 8D shows an example of the deposition of a SiO2 insulation layer;
5. FIG. 8E shows an example of photolithography and etching to open the SiO2 layer as required and to expose the sensors;
6. FIG. 8F shows an example of photolithography to dispose Ag electrodes;
7. FIG. 8G shows an example of the photoresist being removed; and,
8. FIG. 8H shows an example of a completed sensing device with a layer of silicone disposed on top.

FIG. 8I shows an example of how sweat may move through a sensing device 10 that has been formed by the steps in FIGS. 8A to 8H. Typically, sweat moves from an outer body (for example, the skin) of a user into a chamber formed at the inlet of the device 10, around the microfluidic channel and exits the device at the vent/outlet port. Notably, the layers shown in FIG. 8I can be disposed differently, or all features of the device 10 can be formed on one layer or mask.

In particular, it will be appreciated that the embodiment of the device as shown in FIGS. 1A and 1C can allow for minimised layering as the wires from the sensors do not typically cross over the wires from the secondary sensing mechanism.

FIG. 9 shows a photograph of an example of the sensing device 10. In this particular example, the photograph shows two sensing devices formed on a substrate.

It will thus be appreciated that the sweat device described herein can provide data/analytics on the composition of sweat which can assist in determining physiological properties for a user which can then allow for a determination of training regimes to assist the user in achieving higher performance. Further, the sweat device (including the wearable device and the sensing device) allows for a dynamic system that can provide real-time sweat analysis and performance management.

The term “comprise” and variants of that term such as “comprises” or “comprising” are used herein to denote the inclusion of a stated integer or integers but not to exclude any other integer or integers, unless in the context or usage an exclusive interpretation of the term is required.

Those skilled in the art will appreciate that the invention described herein is susceptible to variations and modifications other than those specifically described. All such variations and modifications are to be considered within the scope and spirit of the present invention the nature of which is to be determined from the foregoing description.

1. A measuring device configured to provide an indication of at least one sweat property, the measuring device including:
a sensing device, the sensing device comprising:
an inlet, the inlet being configured to allow sweat to enter the sensing device; and
at least one sensor disposed at least one of at and adjacent to the inlet, the at least one sensor being configured to sense a respective at least one sweat property; and
a wearable device connected to the sensing device, the wearable device being configured to sense skin temperature; and
wherein the sensing device is disposable and interchangeable with another sensing device.

2. The measuring device of claim 1, wherein the at least one sensor comprises at least two sensors disposed at least one of at and adjacent to the inlet, the at least two sensors being configured to sense two sweat electrolytes respectively.

3. The measuring device of claim 1, wherein the at least one sensor comprises at least five sensors disposed at least one of at and adjacent to the inlet, the sensors being configured to sense five respective sweat electrolytes.

4. The measuring device of claim 3, wherein the sweat electrolytes include Sodium ion (Na+), potassium ion (K+), Chloride ion (Cl−), Calcium ions (Ca++), and magnesium ions (Mg++).

5. The measuring device of claim 1, wherein the sensing device includes a secondary sensing mechanism for sensing at least one of sweat volume and sweat rate.

6. The measuring device of claim 5, wherein the secondary sensing mechanism is disposed adjacent to the inlet.

7. The measuring device of claim 5, wherein the secondary sensing mechanism includes a microfluidic channel.

8. The measuring device of claim 7, wherein the microfluidic channel is disposed in a serpentine formation from the inlet to an outlet port.

9. The measuring device of claim 8, wherein the secondary sensing mechanism further includes an electrical impedance sensor.

10. The measuring device of claim 9, wherein the electrical impedance sensor has a body aligned with the microfluidic channel, and two conductors connected to the body at an inlet end and to respective electric pads on the sensing device.

11. The measuring device of claim 10 wherein the two conductors are formed such that they are at least one of spaced-apart and electrically isolated from conductors joining the at least one sensor to the electric pads.

12. The measuring device of claim 11, wherein the two conductors are spaced-apart such that they are substantially parallel at the electric pads.

13. The measuring device of claim 12 wherein the electric pads are positioned at least one of at and adjacent to an edge of the sensing device.

14. The measuring device of claim 1, wherein the sensing device is formed from at least one of a layer and mask that has separate sensing componentry disposed thereon.

15. The measuring device of claim 14, wherein the at least one of a layer and mask includes at least one of:
a first layer having the inlet;
a second layer having at least one opening in fluid communication with the inlet and including at least one sensor for sensing a respective at least one sweat electrolyte;
a third layer having at least one secondary electrode for sensing sweat volume and/or sweat rate; and
a fourth layer having a microfluidic channel.

16. The measuring device of claim 8, wherein the outlet port is a waste chamber and/or hydrophobic vent.

17. (canceled)

18. (canceled)
